(19) United States
(12) Patent Application Publication
(10) Pub. No.: US 2026/0053656 A1
(43) Pub. Date: Feb. 26, 2026

(54) FAST ADJUSTABLE CERVICAL COLLAR

(71) Applicant: Hangzhou Moenmedic Health Technology Co., Ltd, Hangzhou (CN)

(72) Inventor: Junfei Jiang, Hangzhou (CN)

(21) Appl. No.: 18/914,638

(22) Filed: Oct. 14, 2024

(30) Foreign Application Priority Data
Aug. 24, 2024 (CN) 202422066382X

(51) Int. Cl. A61F 5/055

(52) U.S. Cl. CPC A61F 5/055

(57) ABSTRACT

The invention belongs to the technical field of cervical collar, especially refers to a fast adjustable simple cervical collar, including a sponge body and an adjusting bracket located inside the sponge body; the adjusting bracket includes an upper bracket and a lower bracket, the upper bracket and lower bracket realizes distance and angle adjustment between the upper bracket and lower bracket via cooperation of upper bolts, lower bolts and threaded bushings. The adjustment of the cervical collar is faster by adopting the double-bolt structure, which doubles the speed of single-bolt adjustment, and it also realizes adjustment of inclination degree of the cervical collar on left and right sides and height between an upper-layer body and a lower-layer body.

### 1. TECHNICAL FIELD

The invention belongs to the technical field of cervical collars, especially refers to a fast adjustable simple cervical collar.

### 2. BACKGROUND ART

Cervical collar is an auxiliary therapeutic apparatus for cervical spondylosis, which can play the role of braking and protecting the cervical spine, reducing the wear and tear of the nerves, alleviating the traumatic reaction of the intervertebral joints, and facilitating the fading of the tissue edema and consolidation of therapeutic effect, preventing the recurrence of cervical spondylosis; the cervical collar can be applied to all types of cervical spondylosis, for the patients at acute exacerbation stage, especially suitable for the patients with herniation of cervical disc, sympathetic and vertebral artery cervical spondylosis. As people nowadays play with cell phones with their heads down or work in front of computers for a long time, cervical spondylosis is very common.

However, the existing cervical collar shape design is not reasonable enough, which does not have a good ergonomic shape, and the adjustment speed of the cervical collar is slow, thus affecting the user experience, in addition, the existing cervical collar structure is not stable enough and has complex structure, so it's more complicated for the user to use.

For example, a flexible adjustable neck orthosis disclosed on Sep. 1, 2023, with disclosure number CN219614141U, discloses “a flexible adjustable neck orthosis, belongs to the technical field of the neck orthosis, including a shoulder plate and a neck plate, wherein the neck plate is located on top of the shoulder plate, an upper end of the shoulder plate and a lower end of the neck plate are rotatably connected through a pin, upper surfaces at two sides of the shoulder plate are fixedly connected with a second articulating member, lower surfaces at two sides of the neck plate are fixedly connected with a first articulating member, and there is an adjusting mechanism between the first articulating member and the second articulating member. The angle of the neck orthosis can be adjusted according to the patient's situation when the patient is using it, thus avoiding forcible correction of the patient's neck, thus reducing the pain of the patient wearing the orthosis and improving the wearing comfort of patient, and secondly, the patient with uneven shoulder can also be very well corrected when wearing it, thus improving the practicability of the orthosis.” The use of a single screw to adjust has a slow speed, and it uses pins has increased structural complexity, which does not have a good ergonomic shape, etc.

### 3. SUMMARY OF THE INVENTION

The purpose of the invention is to provide a fast adjustable simple cervical collar.

The purpose of the invention is realized as below:
a fast adjustable simple cervical collar includes a sponge body and an adjusting bracket located inside the sponge body; the adjusting bracket includes an upper bracket and a lower bracket, the upper bracket and lower bracket realizes distance and angle adjustment between the upper bracket and lower bracket via cooperation of upper bolts, lower bolts and threaded bushings.

As a further improvement, the upper bracket and lower bracket are integrally molded, an upper side of each upper bolt is connected to the upper bracket, below each upper bolt is provided with a corresponding lower bolt, and a lower side of the lower bolt is connected to the lower bracket; the upper bolt and its corresponding lower bolt below it are threaded in opposite directions, so that when the threaded bushing is rotated, the upper bolt and the lower bolt are close to each other or far away from each other at the same time.

As a further improvement, the upper bolts and lower bolts are symmetrically disposed on left and right sides of the adjusting bracket, wherein the upper bolts and lower bolts are hexagonal bolts, and hexagonal heads of the upper bolts and lower bolts are embedded in hexagonal grooves disposed on the adjusting bracket and are restricted by sealing caps above the hexagonal grooves.

As a further improvement, each sealing cap is in the shape of a rhombus connected to the adjusting bracket by means of screws; a bottom center of the sealing cap is provided with a spherical projection in contact with a center of the corresponding hexagonal head; the upper bolt and lower bolt pass through the adjusting bracket via the through holes provided in the adjusting bracket.

As a further improvement, joints of the upper bracket and lower bracket have good elasticity and toughness and are curved so as to facilitate changing angle and distance between the upper bracket and lower bracket.

As a further improvement, the upper bracket and lower bracket are both U-shaped, width of the joint of the upper bracket and lower bracket is smaller than width of the upper bracket or lower bracket, and width of the lower bracket is larger than width of the upper bracket.

As a further improvement, depth of the U-shape formed by the upper bracket is smaller than depth of the U-shape formed by the lower bracket, the upper bracket is positioned further back relative to the lower bracket, the lower bracket is in the corresponding position of the chest of human body, and the upper bracket is used to support the lower jaw of the human body.

As a further improvement, the adjusting bracket is located in a front part of the sponge body, the adjusting bracket plays the role of a skeleton in the sponge body, and the shape of the front part of the sponge body corresponds to the adjusting bracket.

As a further improvement, the sponge body is separated and disconnected in a middle at a rear of the sponge body, and loop surface hook-and-loop fasteners are provided on left and right sides of the separated and disconnected place at the rear of the sponge body; the left and right loop surface hook-and-loop fasteners and left and right ends of a hook surface hook-and-loop fastener are pasted to each other.

As a further improvement, the sponge body includes an upper-layer body and a lower-layer body, a bottom surface of the lower-layer body being an inwardly concave arc surface, and a top surface of the upper-layer body being an outwardly convex arc surface.

Compared with the prior art, the invention has the following advantages:
(1) The upper bracket and lower bracket realizes distance and angle adjustment between the upper bracket and lower bracket via cooperation of upper bolts, lower bolts and threaded bushings; the adjustment of the cervical collar is faster by adopting the double-bolt structure, which doubles the speed of single-bolt adjustment, and it also realizes adjustment of inclination degree of the cervical collar on left and right sides and height between an upper-layer body and a lower-layer body; in addition, finer adjustment is made possible by the use of bolt adjustments.
(2) The upper bracket and lower bracket are integrally molded, and joints of the upper bracket and lower bracket have good elasticity and toughness and are curved so as to facilitate changing angle and distance between the upper bracket and lower bracket; moreover, the setup of integral molding is a more stable and solid compared to the way of separating and disconnecting the upper bracket and the lower bracket. In addition, the breathability of the cervical collar is great due to the large adjustable space between the upper bracket and lower bracket.
(3) Width of the joint of the upper bracket and lower bracket is smaller than width of the upper bracket or lower bracket, the smaller width of the joint can ensure that the joint has stronger elasticity and toughness; width of the lower bracket is larger than width of the upper bracket, and the wider width of the lower bracket can make the lower bracket support area is larger so as to have more stable support.
(4) The upper bracket and lower bracket are both U-shaped, and depth of the U-shape formed by the upper bracket is smaller than depth of the U-shape formed by the lower bracket, the upper bracket is positioned further back relative to the lower bracket, the lower bracket is in the corresponding position of the chest of human body, and the upper bracket is used to support the lower jaw of the human body, the more ergonomic design making it more comfortable for the user to wear.
(5) Loop surface hook-and-loop fasteners are provided on left and right sides of the separated and disconnected place at the rear of the sponge body, and the left and right loop surface hook-and-loop fasteners and left and right ends of a hook surface hook-and-loop fastener are pasted to each other; the above structure can extend the size of the cervical collar, so that it can be adapted to a more people; in addition, the hook surface hook-and-loop fastener is in detachable connection with the left end or the right end of the loop surface hook-and-loop fasteners so as to facilitate the left-handed or right-handed people to wear.
(6) The sponge body includes an upper-layer body and a lower-layer body, a bottom surface of the lower-layer body being an inwardly concave arc surface, and a top surface of the upper-layer body being an outwardly convex arc surface, making the sponge body more adaptable and more ergonomically designed.
(7) A bottom center of the sealing cap is provided with a spherical projection in contact with a center of the corresponding hexagonal head, and the use of contact between the spherical surface and the flat surface ensures that the upper and lower bolts can oscillate in small increments, thus ensuring that the threaded bushing is not jammed during rotation.

### 4. BRIEF DESCRIPTION OF ACCOMPANY DRAWINGS

FIG. 1 is an overall structural diagram of the invention;

FIG. 2 is a back structural diagram of the invention.

FIG. 3 is a side structural diagram of the invention.

FIG. 4 is a structural diagram of the adjusting bracket provided by the invention.

FIG. 5 is a structural diagram of the sealing cap provided by the invention.

### 5. SPECIFIC EMBODIMENT OF THE INVENTION

The invention is further described in detail hereinafter with reference to the drawings.

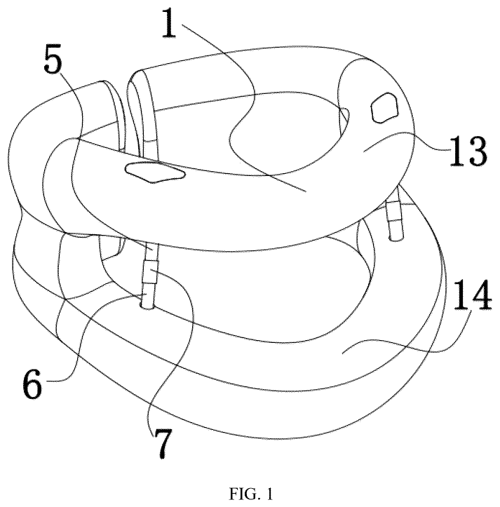

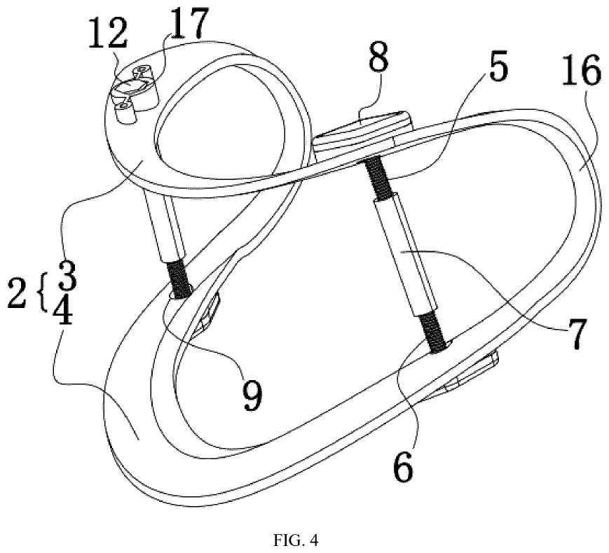

As shown in FIG. 1 and FIG. 4, a fast adjustable simple cervical collar includes a sponge body 1 and an adjusting bracket 2 located inside the sponge body 1; the adjusting bracket 2 includes an upper bracket 3 and a lower bracket 4, the upper bracket 3 and lower bracket 4 realizes distance and angle adjustment between the upper bracket 3 and lower bracket 4 via cooperation of upper bolts 5, lower bolts 6 and threaded bushings 7, which is realized by the following structure, an upper side of each upper bolt 5 is fixedly connected to the upper bracket 3, below each upper bolt 5 is provided with a corresponding lower bolt 6, and a lower side of the lower bolt 6 is connected to the lower bracket 4; the upper bolt 5 and its corresponding lower bolt 6 below it are threaded in opposite directions, so that when the threaded bushing 7 is rotated, the upper bolt 5 and the lower bolt 6 are close to each other or far away from each other at the same time. Thus, it is also possible to extend or shorten the overall length of the upper bolts 5, lower bolts 6 and threaded bushings 7, and then be able to adjust the distance between the upper bracket 3 and the lower bracket 4, that is, to adjust the angle between the upper bracket 3 and the lower bracket 4, which will enable the user to raise or lower the lower jaw, so as to play a good support for the user's lower jaw and head, which is conducive to the user to get a good relaxation and correction of the neck. The adjustment of the cervical collar is faster by adopting the double-bolt structure, which doubles the speed of single-bolt adjustment, and it also realizes adjustment of inclination degree of the cervical collar on left and right sides and height between an upper-layer body 13 and a lower-layer body 14; adjustments can be made to enable the user to raise their head and lift their chin.

The adjustment of the above inclination degree is mainly through the following specific adjustment: when the user's neck or head is tilted to the left, the user can adjust the overall height of the upper bolt 5, lower bolt 6 and the threaded bushing 7 on the left side of the sponge body 1 to be higher, and make the corresponding height of the right side lower than that of the left side, so as to realize the correction of the tilted posture of the user's neck or head. Conversely, when the user's neck or head is tilted toward the right side, the overall height of the upper bolt 5, the lower bolt 6, and the threaded bushing 7 on the right side of the sponge body 1 can be adjusted upward and the height of the corresponding left side can be lowered compared to that of the right side, so that the correction of the user's posture can be realized; moreover, the cervical collar structure is made more stable and simple because no excessive connecting parts, such as pins, are used.

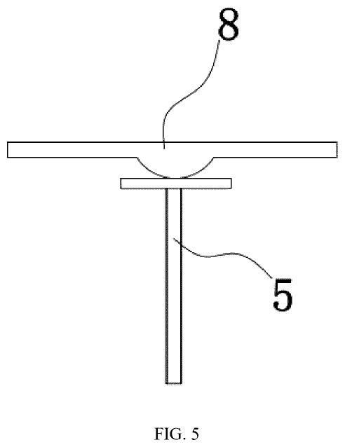

As shown in FIG. 4 and FIG. 5, the upper bolts 5 and lower bolts 6 are symmetrically disposed on left and right sides of the adjusting bracket 2, wherein the upper bolts 5 and lower bolts 6 are hexagonal bolts, and hexagonal heads of the upper bolts 5 and lower bolts 6 are embedded in hexagonal grooves 17 disposed on the adjusting bracket 2 and are restricted by sealing caps 8 above the hexagonal grooves 17; each sealing cap 8 is connected to the adjusting bracket 2 by means of screws; a bottom center of the sealing cap 8 is provided with a spherical projection in contact with a center of the corresponding hexagonal head 12; the upper bolt 5 and lower bolt 6 pass through the adjusting bracket 2 via the through holes 9 provided in the adjusting bracket 2; the hexagonal grooves 17 play a role in restricting the rotation of the hexagonal heads 12 on the upper bolts 5 and the lower bolts 6, and then the hexagonal heads 12 are fixed in the hexagonal grooves 17 by the spherical protrusions of the sealing caps 8 to restrict the upward and downward movement thereof, but due to the contact limitation method between the spherical surface and the flat surface, the hexagonal heads 12 are not completely fixed, and can be rotated in a small extent relative to the above-mentioned spherical surface. The opening angle of the adjusting bracket 2 is changing during the adjustment process, if the upper bolt 5 and the lower bolt 6 are completely fixed, it will result in the upper bolt 5 and the lower bolt 6 and the threaded bushing 7 being jammed in the rotating and adjusting process and the three of them will not be able to rotate smoothly, so through the above mentioned technical solution, it is crucial to design the lower bolt 6 and the threaded bushing 7 to be able to swing in a small range.

As shown from FIG. 1 to FIG. 3, the upper bracket 3 and lower bracket 4 are integrally molded, and joints of the upper bracket 3 and lower bracket 4 have good elasticity and toughness and are curved so as to facilitate changing angle and distance between the upper bracket 3 and lower bracket 4; the upper bracket 3 and lower bracket 4 are both U-shaped, width of the joint of the upper bracket 3 and lower bracket 4 is smaller than width of the upper bracket 3 or lower bracket 4, and width of the lower bracket 4 is larger than width of the upper bracket 3, and the wider width of the lower bracket 4 compared to the upper bracket 3 can make the lower bracket 4 support area larger so as to have more stable support.

Depth of the U-shape formed by the upper bracket 3 is smaller than depth of the U-shape formed by the lower bracket 4, the upper bracket 3 is positioned further back relative to the lower bracket 4, the lower bracket 4 is in the corresponding position of the chest of human body, and the upper bracket 3 is used to support the lower jaw of the human body. After wearing the cervical collar, this structural design of the cervical collar allows the user's neck and head to move backward and upward after wearing, thus providing a good relaxation and corrective effect, which is more in line with the ergonomic design and makes it more comfortable for the user to wear.

As shown from FIG. 1 to FIG. 4, the adjusting bracket 2 is located in a front part of the sponge body 1, the adjusting bracket 2 plays the role of a skeleton in the sponge body 1, and the shape of the front part of the sponge body 1 corresponds to the adjusting bracket 2.

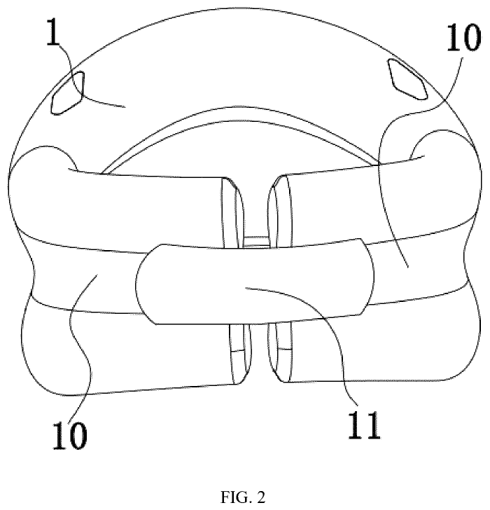

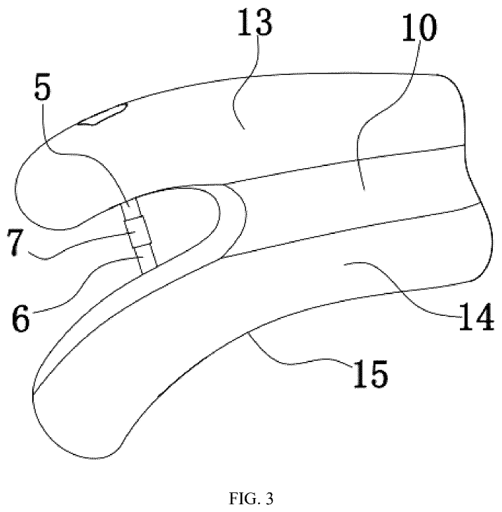

As shown in FIG. 2, the sponge body 1 is separated and disconnected in a middle at a rear of the sponge body 1, and loop surface hook-and-loop fasteners 10 are provided on left and right sides of the separated and disconnected place at the rear of the sponge body 1; the left and right loop surface hook-and-loop fasteners 10 and left and right ends of a hook surface hook-and-loop fastener 11 are pasted to each other. Since the neck size for different people are different, it can be adapted to fit different people through connection method of the hook-and-loop fasteners; as shown in FIG. 3, the sponge body 1 includes an upper-layer body 13 and a lower-layer body 14, a bottom surface of the lower-layer body 14 being an inwardly concave arc surface 15, and a top surface of the upper-layer body 13 being an outwardly convex arc surface 16; wherein the concave arc surface 15 is set to allow the lower-layer body 14 to adapt to different shapes and sizes of the breasts of different people, in line with the ergonomic shape design, thereby making the user more comfortable to wear.

The invention and its embodiments have been described above, but the description is not limited thereto; only one embodiment of the invention is shown in the drawings, and the actual structure is not limited thereto. In general, it is to be understood by those skilled in the art that non-creative design of structural forms and embodiments that are similar to the technical solutions without departing from the spirit of the invention shall all fall within the protective scope of the invention.

1. A fast adjustable simple cervical collar, including a sponge body and an adjusting bracket located inside the sponge body; the adjusting bracket includes an upper bracket and a lower bracket, the upper bracket and lower bracket realizes distance and angle adjustment between the upper bracket and lower bracket via cooperation of upper bolts, lower bolts and threaded bushings.

2. The adjustable simple cervical collar of claim 1, wherein the upper bracket and lower bracket are integrally molded, an upper side of each upper bolt is connected to the upper bracket, below each upper bolt is provided with a corresponding lower bolt, and a lower side of the lower bolt is connected to the lower bracket; the upper bolt and its corresponding lower bolt below it are threaded in opposite directions, so that when the threaded bushing is rotated, the upper bolt and the lower bolt are close to each other or far away from each other at the same time.

3. The adjustable simple cervical collar of claim 2, wherein the upper bolts and lower bolts are symmetrically disposed on left and right sides of the adjusting bracket, wherein the upper bolts and lower bolts are hexagonal bolts, and hexagonal heads of the upper bolts and lower bolts are embedded in hexagonal grooves disposed on the adjusting bracket and are restricted by sealing caps above the hexagonal grooves.

4. The adjustable simple cervical collar of claim 3, wherein each sealing cap is in the shape of a rhombus connected to the adjusting bracket by means of screws; a bottom center of the sealing cap is provided with a spherical projection in contact with a center of the corresponding hexagonal head; the upper bolt and lower bolt pass through the adjusting bracket via the through holes provided in the adjusting bracket.

5. The adjustable simple cervical collar of claim 1, wherein joints of the upper bracket and lower bracket have good elasticity and toughness and are curved so as to facilitate changing angle and distance between the upper bracket and lower bracket.

6. The adjustable simple cervical collar of claim 4, wherein the upper bracket and lower bracket are both U-shaped, width of the joint of the upper bracket and lower bracket is smaller than width of the upper bracket or lower bracket, and width of the lower bracket is larger than width of the upper bracket.

7. The adjustable simple cervical collar of claim 5, wherein depth of the U-shape formed by the upper bracket is smaller than depth of the U-shape formed by the lower bracket, the upper bracket is positioned further back relative to the lower bracket, the lower bracket is in the corresponding position of the chest of human body, and the upper bracket is used to support the lower jaw of the human body.

8. The adjustable simple cervical collar of claim 1, wherein the adjusting bracket is located in a front part of the sponge body, the adjusting bracket plays the role of a skeleton in the sponge body, and the shape of the front part of the sponge body corresponds to the adjusting bracket.

9. The adjustable simple cervical collar of claim 1, wherein the sponge body is separated and disconnected in a middle at a rear of the sponge body, and loop surface hook-and-loop fasteners are provided on left and right sides of the separated and disconnected place at the rear of the sponge body; the left and right loop surface hook-and-loop fasteners and left and right ends of a hook surface hook-and-loop fastener are pasted to each other.

10. The adjustable simple cervical collar of claim 1, wherein the sponge body includes an upper-layer body and a lower-layer body, a bottom surface of the lower-layer body being an inwardly concave arc surface, and a top surface of the upper-layer body being an outwardly convex arc surface.
